(19) United States
(12) Patent Application Publication
(10) Pub. No.: US 2005/0203056 A1
(43) Pub. Date: Sep. 15, 2005

(54) Carboxyl-reduced derivatives of hyaluronic acid, preparation thereof, use thereof as a medicinal product and the pharmaceutical compositions containing them

(75) Inventors: Wolfgang Ulmer, Eppstein (DE); Christian Viskov, Ris Orangis (FR); Philippe Hubert, Maisons-Alfort (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 11/015,947

(22) Filed: Dec. 17, 2004

Related U.S. Application Data
(60) Provisional application No. 60/618,661, filed on Oct. 12, 2004.

(30) Foreign Application Priority Data
Dec. 19, 2003 (FR) 0314989

(51) Int. Cl. A61K 31/728; C08B 37/00

(52) U.S. Cl. 514/54; 536/53

(57) ABSTRACT

The present invention relates to the carboxyl-reduced derivatives of hyaluronic acid of formula (I): 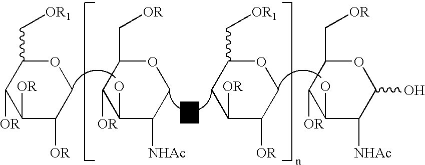
in which R or R₁ represents H or SO₃M, n is an integer of between 0 and 25 000, M is an alkali metal, isolated or as mixtures, to the diastereoisomers thereof, to the process for the preparation thereof, to the uses thereof as a medicinal product and to the pharmaceutical compositions containing them.

This application claims the benefit of U.S. Provisional Application No. 60/618,661 filed Oct. 12, 2004 and benefit of priority from French Patent Application No. 03 14989, filed Dec. 19, 2003, both of which are incorporated herein by reference in their entirety.

The present invention relates to novel glycosaminoglycans and also to their pharmaceutically acceptable addition salts, and more precisely to carboxyl-reduced and chemoselectively O-sulfated derivatives (compounds) of hyaluronic acid, isolated or as a mixture, to the use thereof as a medicinal product and to the pharmaceutical compositions containing them.

Glycosaminoglycans (GAGs) are essentially made up of alternating uronic acid-amino sugar (or vice versa) units of the type such as those encountered in the oligosaccharide or polysaccharide chains of biologically active natural GAGs such as heparin, heparan sulfate, dermatan sulfate, chondroitins, chondroitin sulfates or hyaluronic acid.

The uronic acid units correspond more specially to the D-glucoronic or L-iduronic acid structure and the amino sugar units to the D-glucosamine or D-galactosamine structure.

The natural GAGs exhibit therapeutic activities as thrombin inhibitors. They therefore exhibit anti-thrombotic and anticoagulant activity and are used in cardiovascular pathologies in which there is a risk of thrombosis.

O-persulfated derivatives of hyaluronic acid have been described and studied for their anticoagulant properties (JP200080102-A).

N,O-sulfated derivatives of hyaluronic acid have been described and studied for their antiulcer properties (WO 00/01394-A1) or their anticoagulant properties (WO 99/43728-A1), (WO 98/45335-A1).

Similarly sulfated derivatives of hyaluronic acid are described for their anticoagulant properties (FR 2584728).

Moreover, O-persulfated derivatives of hyaluronic acid have been described and studied for their properties in rheumatoid arthritis (WO 92/13541-A1).

The subject of the present invention is a novel process using a carboxyl reduction step and a sulfatation (sulfating) step for obtaining novel hyaluronic acid derivatives exhibiting advantageous properties for treating or preventing disorders which display an increased activity of at least one of the matrix metalloproteinases.

The carboxyl-reduced and chemoselectively O-sulfated derivatives of hyaluronic acid of formula (I) according to the invention have a very regular polymeric structure with degrees of purity of the order of 90%. Reproducible and unexpected biological properties result therefrom.

In the pathological condition of osteoarthritis, the degradation of aggrecan, the main proteoglycan of cartilage in the joints, represents a very early and crucial event. The pathological loss of aggrecan is caused by proteolytic cleavages in its interglobular domain. Amino acid sequence analyses of the proteoglycan metabolites isolated from the synovial fluid of patients suffering from joint damage, from osteoarthritis or from an inflammatory disorder of the joints, have shown that proteolytic cleavage exists between the amino acids Glu³⁷³ and Ala³⁷⁴ in the interglobular domain of human aggrecan (Lohmander, et al., Arthritis Rheum., 36: 1214-1222 (1993)). The proteolytic activity responsible for this cleavage is called “aggrecanase” and can be attributed to the metalloproteinase (MP) or matrix metalloproteinase (MMP) superfamily.

Zinc is an essential element in the catalytically active center of metalloproteinases. MMPs cleave collagen, laminin, proteoglycans, elastin or gelatin under physio-logical conditions. They therefore play an important role in bone tissue and connective tissue. A large number of different MMP inhibitors are known (see, for example, patent applications EP 0 606 046 or WO 94/28889). However, the known MMP inhibitors frequently have a significant disadvantage. They lack specificity for any particular class of MMP. On the contrary, most MMP inhibitors simultaneously inhibit a plurality of MMPs.

Consequently, a need exists for MMP inhibitors that have more narrowly defined specificities in order to more effectively treat or prevent specific disorders.

A subject of the invention is most particularly the carboxyl-reduced derivatives of hyaluronic acid of formula (I): 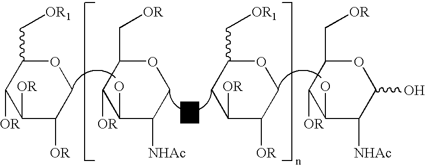
in which R represents SO₃M, and R₁ represents H or SO₃M, n is an integer of between 0 and 25 000, M is an alkali metal, said derivatives being in the form of an isolated compound or in the form of mixtures, and also the diastereoisomers thereof.

In particular, a subject of the invention is the carboxyl-reduced derivatives of hyaluronic acid according to formula (I), wherein M is chosen from sodium, calcium, magnesium and potassium.

According to a preferred aspect of the invention, M is a sodium atom.

A subject of the invention is more particularly the carboxyl-reduced derivatives of hyaluronic acid as described above, wherein R and R₁ represent SO₃Na.

A subject of the invention is more particularly the carboxyl-reduced derivatives of hyaluronic acid as described above, wherein R represents SO₃Na and R₁ represents H.

The polysaccharides according to the invention thus comprise an even number of saccharides.

The persulfated carboxyl-reduced derivatives of hyaluronic acid according to the invention are obtained according to the process using successively the following steps:
trans-salification (salt exchange) of hyaluronic acid,
sulfatation in organic medium of the trans-salified hyaluronic acid, followed by salification,
carboxyl reduction of the persulfated derivative
   either a) by means of a carbodiimide derivative in the presence of a reducing agent,
   or b) by esterification, the persulfated derivative being, where appropriate, trans-salified beforehand with a quaternary ammonium salt, followed by reduction of the corresponding ester derivative by the action of a reducing agent, then
where appropriate, trans-salification of the persulfated, carboxyl-reduced derivative with a quaternary ammonium salt, and then resulfatation (resulfating) followed by salification (salt formation).

The following reaction scheme illustrates the present invention:

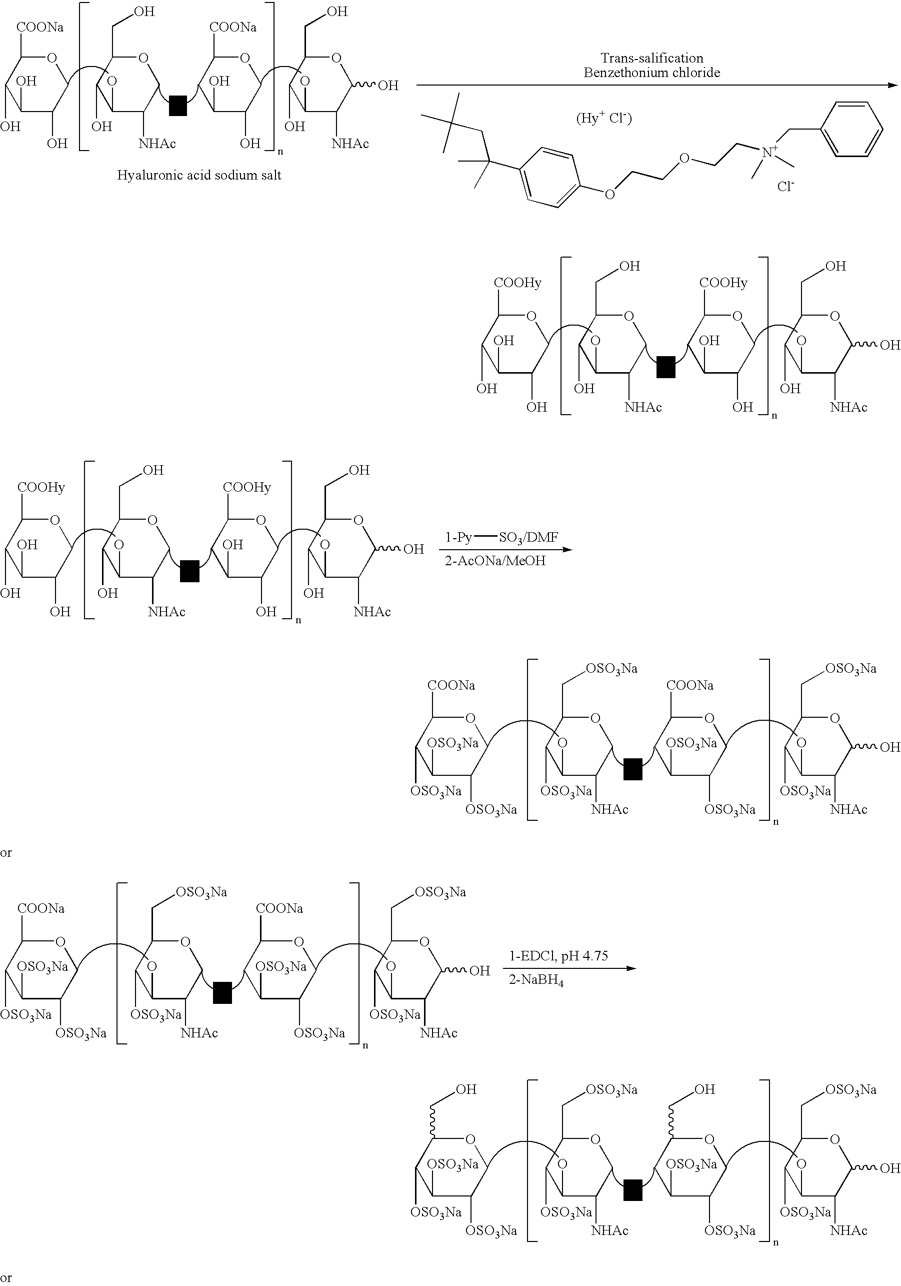

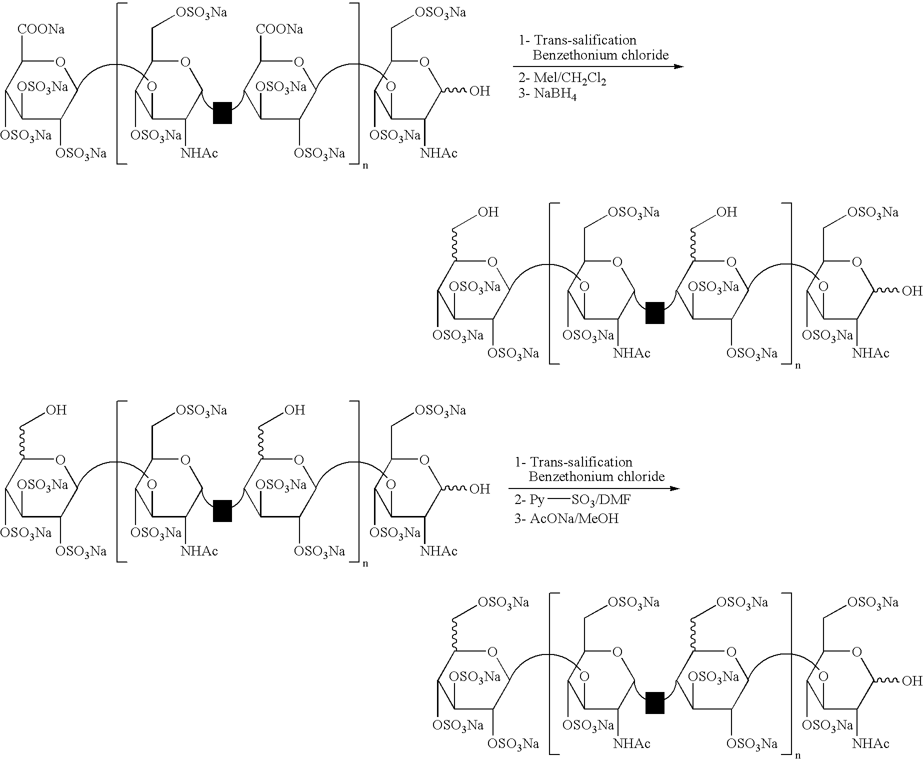

The process according to the present invention is characterized by the strong chemoselectivity of the carboxyl reduction and sulfatation reactions. The products which result therefrom are notably homogeneous and result in true polymers. The carboxyl-reduced hyaluronic acid derivatives have purities of the order of 90%. This homogeneity is determined by NMR and infrared structural analysis.

Reproducible and unexpected biological properties result therefrom.

The persulfatation (persulfation) reactions are preferably carried out in organic medium by means of a complex of sulfuric anhydride with an organic base such as pyridine or trimethylamine. They are generally followed by a salification reaction, for example by the action of sodium acetate.

For optimal chemoselectivity of the persulfation reactions, it is preferable to use a benzethonium salt in the presence of 10 to 30 equivalents of pyridine-sulfuric anhydride complex per hydroxyl function to be sulfated. Similarly, the reaction will preferably be carried out at temperatures of between 10 and 70° C.

The persulfatation of the hyaluronic acid benzethonium salt is most particularly carried out at between 50 and 70° C. This is illustrated in example 2a or 2b.

During the sulfatation of the sulfated, carboxyl-reduced derivative, benzethonium salt (compounds of formula (I) with R₁═H and R═SO₃Na, second sulfation), the procedure is preferably carried out in organic medium by means of a complex of sulfuric anhydride with an organic base such as pyridine, at temperatures in the region of 20° C. This is illustrated in example 3.

The carboxyl reduction of hyaluronic acid derivatives is carried out in the presence of a carbodiimide derivative. By way of example, it is possible to use 1-ethyl-3-(3-dimethylaminopropyl)carbodiimide hydrochloride.

The term “derivatives (compounds) of hyaluronic acid” is intended to mean the persulfated derivatives of hyaluronic acid, the hyaluronic acid benzethonium salt, the persulfated derivatives of hyaluronic acid benzethonium salts and also the hyaluronic acid sodium salt.

The reduction per se is preferably carried out with an alkali metal borohydride. By way of example, the reaction with the carbodiimide derivative is carried out with sodium borohydride.

The reaction for formation of the adduct with the carbodiimide derivative is preferably carried out in the presence of 5 to 20 equivalents of reactant and at a pH of between 4 and 5.

Even more preferably, the reaction with the carbodiimide derivative is carried out in the presence of 7 to 13 equivalents of reactant and at a pH of between 4.3 and 4.9.

The reduction per se of the activated adduct of the hyaluronic acid derivative is carried out with 10 to 300 equivalents of alkali metal borohydride at a temperature of between 10 and 70° C. More preferably, the reduction of the abovementioned adduct is carried out in the presence of 140 to 250 equivalents of alkali metal borohydride at a temperature of between 20 and 50° C.

According to another embodiment of the present invention, the carboxyl reduction can be carried out by reduction of an ester of the hyaluronic acid derivatives. By way of example, it is possible to use a methyl ester derivative. The hyaluronic acid, trans-salified beforehand in the form of the benzethonium salt, is esterified by the conventional methods of esterification known to those skilled in the art, using an alkyl halide containing from 1 to 6 carbon atoms, such as methyl iodide, or an arylalkyl halide, such as benzyl chloride, in organic medium. The esterification per se of the hyaluronic acid benzethonium salt derivative is preferably carried out in dichloromethane in the presence of 2 to 20 equivalents of methyl iodide and at a temperature in the region of 20° C.

In particular, the esterification is carried out in dichloromethane in the presence of 4 to 10 equivalents of methyl iodide for approximately 6 days and at a temperature in the region of 20° C.

The reduction per se of the methyl ester of the hyaluronic acid derivative is carried out with 10 to 300 equivalents of alkali metal borohydride at a temperature of between 10 and 70° C. By way of example, the reduction of the methyl ester is carried out with sodium borohydride.

More preferably, the reduction of the abovementioned methyl ester is carried out in the presence of 140 to 250 equivalents of alkali metal borohydride at a temperature in the region of 20° C.

In particular, a subject of the invention is the process using the following steps:
trans-salification of hyaluronic acid with benzethonium chloride,
sulfatation of a quaternary ammonium salt of hyaluronic acid in organic medium by means of a complex of sulfuric anhydride with pyridine or trimethylamine, followed by salification with sodium acetate,
   either a) carboxyl reduction by means of 1-(3-dimethylaminopropyl)-3-ethylcarbodiimide hydrochloride in the presence of sodium borohydride,
   or b) esterification by the action of methyl iodide on the hyaluronic acid derivative trans-salified beforehand with benzethonium chloride, then reduction of the corresponding methyl ester with sodium borohydride,
   where appropriate, trans-salification of the sulfated, carboxyl-reduced derivative with benzethonium chloride, then resulfatation with the sulfuric anhydride-organic base complex, followed by salification with sodium acetate.

When it is desired to obtain isolated derivatives from the mixture of carboxyl-reduced derivatives of hyaluronic acid as obtained according to the process described above, the following process should then be applied to the mixture:
fractionation of the mixture by chromatography on columns filled with gel of polyacrylamide agarose type or a polyacrylamide gel. The mixture is eluted with a sodium hydrogen carbonate solution.

The sodium hydrogen carbonate solution is preferably a solution of 0.1 to 1 mol/l. Even more preferably, the separation is carried out at a concentration of 1 mol/l. The detection is carried out by refractometry.

A subject of the invention is also the carboxyl-reduced and chemoselectively O-sulfated derivatives of hyaluronic acid, isolated or as a mixture as defined above, which can be obtained according to the process(es) as defined above.

The polysaccharides of formula (I), isolated or as mixtures, can be used as medicinal products.

These polysaccharides exhibit in particular strong inhibitory activity on certain matrix metalloproteases. These inhibitors are particularly indicated for the treatment of pathological states where a large increase in matrix metalloproteinase activity is noted.

The pathological states to which the present invention refers involve an increase in the activity of at least one of the following matrix metalloproteinases: neutrophil elastase, matrilysin (MMP-7), aggrecanase, hADAMTS1 and gelatinase A (MMP-2).

The compounds can therefore be used for preventing and treating diseases such as degenerative joint disorders (such as osteoarthritis), spondylosis, chondrolysis associated with joint trauma or prolonged joint immobilizations (often following an injury to the meniscus or patella or the rupture of ligaments), disorders related to injuries, periodontal disorders, chronic disorders of the locomotor system (such as chronic or acute inflammatory, immunological or metabolic forms of arthritis), arthropathies, myalgias, or disorders related to bone metabolism.

A subject of the invention is also the pharmaceutical compositions containing the compounds of formula (I), isolated or as a mixture, and also one or more pharmaceutically acceptable excipients, vehicles or additives.

Another aspect of the invention is a process for preparing the pharmaceutical compositions containing the compounds of formula (I), wherein an amount corresponding to a desired dose of a compound of formula (I) is mixed with one or more pharmaceutically acceptable excipients, vehicles, additives.

The compounds of formula (I) can be administered via various routes. They may include, without being limited, subcutaneous, intraarticular, intraperitoneal or intravenous injections.

The administration may also be rectal, oral, by inhalation, or else transdermal.

In the case of solutions for injection (for example in the form of an ampoule), the doses may range from 5 μg to approximately 200 mg of compound of formula (I), and preferably from 10 μg to 40 mg.

The daily dose indicated for the treatment of an adult patient weighing approximately 70 kg is from 10 μg to 500 mg of active ingredients, generally from 20 mg to approximately 100 mg. However, depending on the circumstances, higher or lower daily doses may be appropriate.

These doses can be administered once a day in the form of a single dosage unit.

Alternatively, the doses can be administered in a plurality of smaller doses, given repeatedly at defined intervals over time.

The following examples illustrate the invention without, however, limiting it.

The hyaluronic acid used for preparing the compounds illustrating this invention has a molecular mass of 2.7×10⁶ Daltons. It is prepared and marketed by the company CAREF under the name hyaluronate Na F100.

### EXAMPLE 1

### Preparation of the Carboxyl-Reduced Hyaluronic Acid Sodium Salt (Without the Essential Step of Sulfatation)

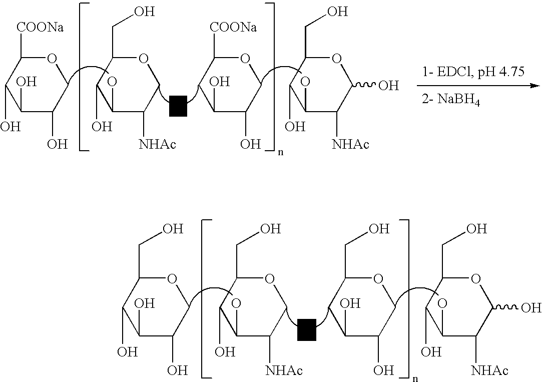 (Polysaccharide of formula (I) with n=0 to 25 000, R=R1=H)

A solution of 1 g of hyaluronic acid sodium salt in 150 ml of water is prepared at a temperature in the region of 20° C. The pH is adjusted to pH 4.7±0.1 with a 0.1 N hydrochloric acid solution. 4.48 g of 1-ethyl-3-(3-dimethylaminopropyl)carbodiimide hydrochloride are added and the mixture is stirred while maintaining the pH at 4.7±0.1. When the pH no longer changes, 17.89 g of sodium borohydride and 250 ml of water are then added in small portions. The reaction medium is stirred for approximately 2 hours at a temperature in the region of 50° C. After cooling to a temperature in the region of 5° C., the reaction medium is neutralized with hydrochloric acid at a pH in the region of 7 (volume of acid 160 ml). The white suspension is loaded into an MW 10 000 cellulose ester membrane and dialyzed, changing the water baths regularly for 72 hours (the third bath is effected with a 0.2 N sodium chloride solution). The content of the membranes is lyophilized. 0.86 g of a white lyophilized material is obtained. The yield obtained is 95%.

Proton spectrum in D₂O, 400 MHz, T=333 K (δ in ppm—mixture of C5 epimers): 2.0 (3H, s), 3.3 (1H, m), between 3.4 and 3.55 (5H, m), 3.6 (1H, d, J=7 Hz, between 3.63 and 3.85 (4H, m), 3.89 (1H, d, J=7 Hz), 4.45 (1H, s), 4.57 (1H, m).

### EXAMPLE 2A

### Preparation of the Persulfated, Carboxyl-Reduced Hyaluronic Acid Sodium Salt

(Polysaccharide of formula (I) with n=0 to 25 000, R═SO₃Na, R1=H) 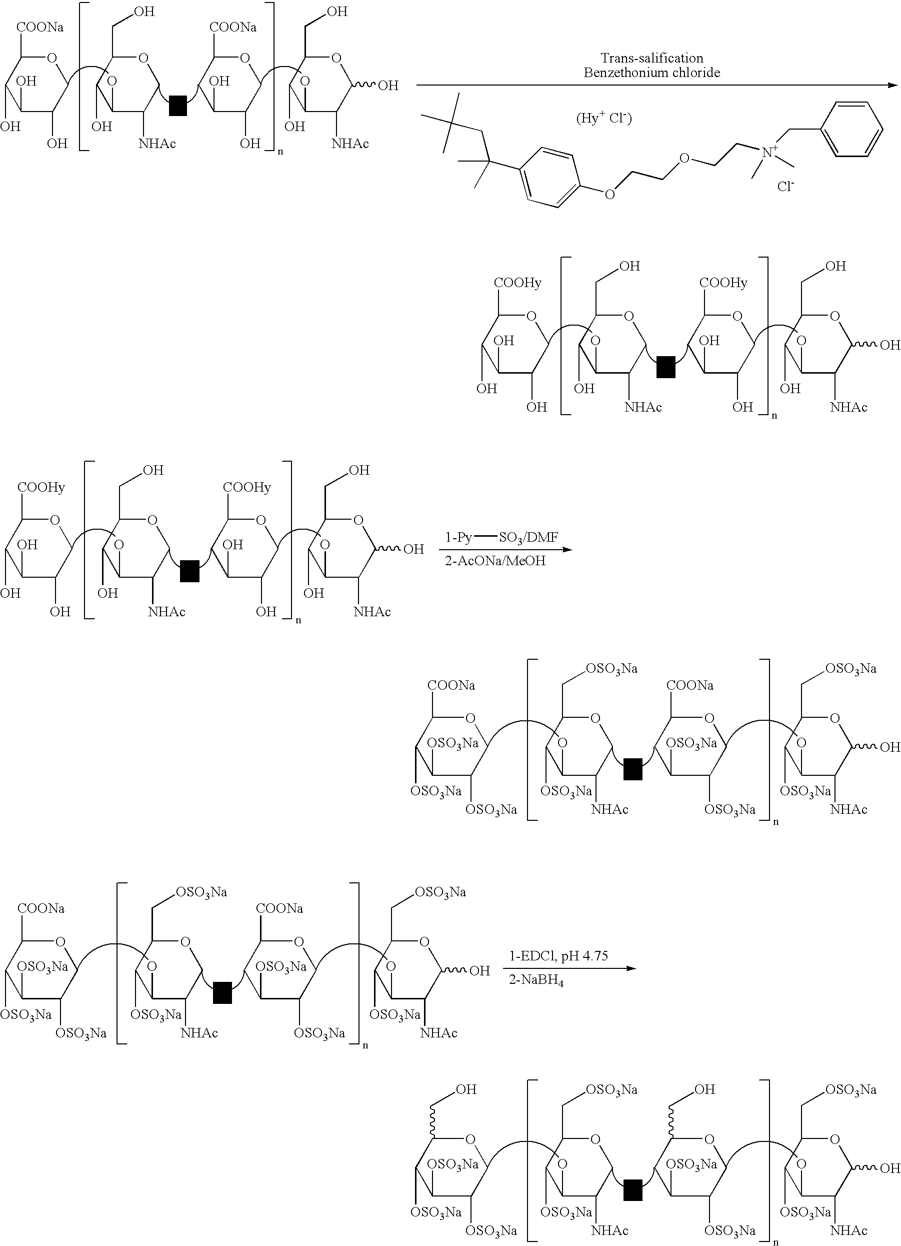
a) Preparation of the Hyaluronic Acid Benzethonium Salt (Trans-Salification)

A solution of 23.5 g of benzethonium chloride in 200 ml of water is added to a solution of 20 g of hyaluronic acid sodium salt in 3.6 l of water, over approximately 20 minutes. A white precipitate forms. After stirring for 1 h, the mixture is allowed to sediment for approximately 1 h and the supernatant is discarded and then replaced with the same amount of water (2.8 l). After stirring for 1 h, the mixture is allowed to sediment for approximately 1 h and the supernatant is discarded and then replaced with the same amount of water (3 l). After stirring for 1 h, the mixture is allowed to sediment for approximately 1 h and the supernatant is discarded and then replaced with the same amount of water (3.2 l). The product is filtered, washed with three portions of 1 l of water, and then dried on a screen. The product is subsequently ground and then dried for approximately 48 h at a temperature in the region of 50° C. under reduced pressure (6 kPa). 35.6 g of hyaluronic acid benzethonium salt are obtained. The reaction yield is quantitative.

b) Preparation of the Persulfated Hyaluronic Acid Sodium Salt (Sulfatation)

3 g of hyaluronic acid benzethonium salt are dissolved in 240 ml of anhydrous dimethylformamide at a temperature in the region of 60° C. and under an inert atmosphere. A solution of 36.34 g of pyridine-sulfuric anhydride complex in 210 ml of anhydrous dimethylformamide is added to the solution obtained. After stirring for 3 hours at a temperature in the region of 60° C., the reaction medium is cooled to a temperature in the region of −5° C. A mixture of 225 ml of water and 1350 ml of a 10% sodium acetate solution in methanol is added to the reaction medium. The reaction medium is filtered and the cake is washed with three portions of 60 ml of methanol. The solid obtained is distilled in water and is then loaded into a MW 10 000 cellulose ester membrane and dialyzed, changing the water baths regularly for 72 hours (the second bath is effected in a 0.2 N sodium chloride solution). The content of the membrane is lyophilized. 2.65 g of a white lyophilized material are obtained. The yield obtained is 86%.

Proton spectrum in D₂O, 400 MHz, T=298 K, δ in ppm: 2.1 (3H, s), 3.85 (2H, m), 4.07 (1H, m), 4.17 (2H, m), 4.38 (1H, m), 4.42 (2H, m), 4.55 (1H, d, J=7 Hz), 4.8 (2H, m), 4.88 (1H, m).

c) Preparation of the Persulfated, Carboxyl-Reduced Hyaluronic Acid Sodium Salt (Carboxyl Reduction)

A solution of 0.5 g of persulfated hyaluronic acid sodium salt in 30 ml of water is prepared. The pH is adjusted to 4.7±0.1 with a 0.1 N hydrochloric acid solution. 1.11 g of 1-ethyl-3-(3-dimethylaminopropyl)carbodiimide hydrochloride are added and the mixture is then stirred while maintaining the pH at 4.7±0.1. When the pH no longer changes, 4.44 g of sodium borohydride are then added in small portions. The reaction medium is stirred for approximately 2 hours at a temperature in the region of 50° C. After cooling to a temperature in the region of 10° C., the reaction medium is neutralized at pH 7 with concentrated hydrochloric acid (12 N). The white suspension is loaded into a MW 10 000 cellulose ester membrane and dialyzed, changing the water baths regularly for 72 hours (the second bath is effected with a 0.2 N sodium chloride solution). The content of the membrane is lyophilized. 0.36 g of a white lyophilized material is obtained. The yield obtained is 76%.

Proton spectrum in D₂O, 400 MHz, T=333 K, δ in ppm: 2.0 (3H, s), between 3.6 and 3.72 (3H, m), 3.8 (1H, t, J=6 Hz), 4.05 (2H, m), 4.13 (1H, dd, J=7 and 3 Hz), 4.2 (1H, t, J=7 Hz), 4.4 (1H, t, J=3 Hz), 4.48 (1H, d, J=8 Hz), 4.6 (1H, t, J=3 Hz), 4.75 (1H, d, J=7 Hz), 4.82 (1H, d, J=3 Hz).

### EXAMPLE 2B

### Preparation of the Persulfated, Carboxyl-Reduced Hyaluronic Acid Sodium Salt

(Polysaccharide of formula (I) with n=0 to 25 000, R═SO₃Na, R1=H) 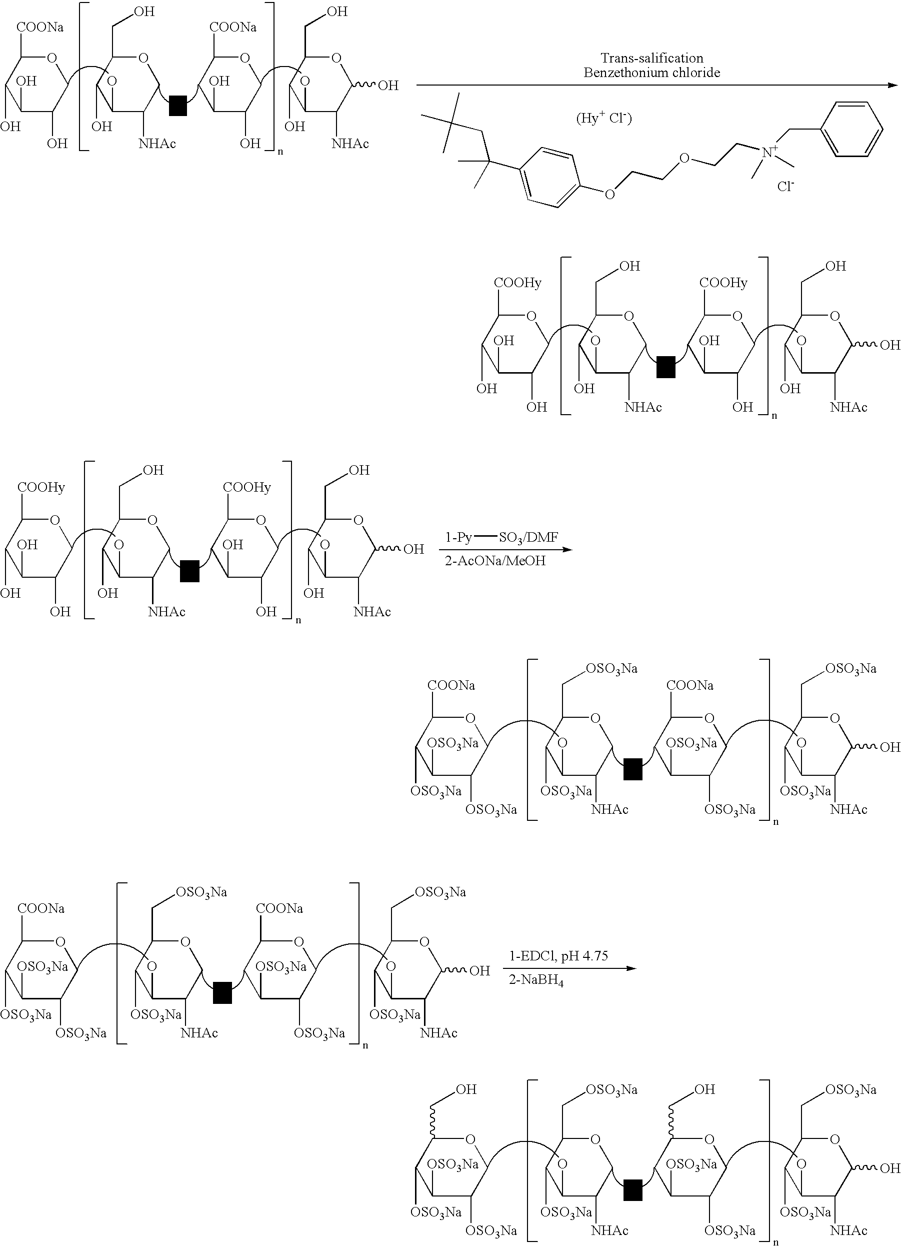
a) Preparation of the Hyaluronic Acid Benzethonium Salt (Trans-Salification)

The hyaluronic acid benzethonium salt is prepared according to example 2a.

b) Preparation of the Persulfated Hyaluronic Acid Sodium Salt (Sulfatation)

The persulfated hyaluronic acid sodium salt is prepared according to example 2a.

c) Preparation of the Persulfated Hyaluronic Acid Benzethonium Salt (Trans-Salification)

A solution of 5.6 g of benzethonium chloride in 100 ml of water is added to a solution of 1.96 g of persulfated hyaluronic acid sodium salt in 115 ml of water, over approximately 5 minutes. A white precipitate forms. After stirring for 1 h, the mixture is allowed to sediment overnight. The product is filtered, washed with two portions of 80 ml of water and then dried in a desiccator under vacuum. 5.3 g of persulfated hyaluronic acid benzethonium salt are obtained. The reaction yield is 80%.

d) Preparation of the Methyl Ester of the Persulfated Hyaluronic Acid Sodium Salt

2.7 g of molecular sieve 4 Å are added to a solution of 2.76 g of persulfated hyaluronic acid benzethonium salt in 14.7 g of dichloromethane. The mixture is stirred for 3 h 30 min. The molecular sieve is separated from the solution, and rinsed with 2.3 g of dichloromethane. 2.4 g of methyl iodide are added to the anhydrous solution of persulfated hyaluronic acid benzethonium salt in dichloromethane. The mixture is stirred at a temperature in the region of 20° C. for 6 days. The mixture is added to 45 ml of a 10% sodium acetate solution in methanol. After stirring for 1 h, the mixture is allowed to sediment for approximately 1 h and the supernatant is discarded and then replaced with the same amount of methanol (35 ml). After stirring for 15 min, the product is filtered, washed with methanol (twice 5 ml), dried in a desiccator and then dried at a temperature in the region of 50° C. under reduced pressure (6 kPa). 0. 5 g of methyl ester of the persulfated hyaluronic acid sodium salt is obtained. The reaction yield is 78%.

Proton spectrum in D₂O, 400 MHz, T=298 K, δ in ppm: 2.0 (3H, s), 3.68 (1H, m), 3.71 (3H, s), 3.80 (1H, m), 4.05 (2H, m), 4.22 (1H, m), 4.26 (1H, m), 4.35 (1H, m), between 4.4 and 4.55 (2H, m), 4.70 (2H, m), 4.83 (1H, m).

e) Preparation of the Persulfated, Carboxyl-Reduced Hyaluronic Acid Sodium Salt (Carboxyl Reduction)

0.93 g of NaBH₄ is added, in small portions, at a temperature in the region of 20° C., to a solution of 0.1 g of methyl ester of the persulfated hyaluronic acid sodium salt in 6 ml of water. The mixture obtained is stirred at a temperature in the region of 20° C. for approximately 18 h. After cooling to a temperature in the region of 5° C., the reaction medium is neutralized at pH 7 with concentrated hydrochloric acid (12 N). The mixture obtained is loaded into an MW 10 000 cellulose ester membrane and dialyzed, changing the water baths regularly for 4 days. The content of the membrane is lyophilized. 44 mg of a white lyophilized material are obtained. The yield obtained is 44%.

Proton spectrum in D₂O, 400 MHz, T=333 K, δ in ppm: 2.0 (3H, s), between 3.6 and 3.72 (3H, m), 3.8 (1H, t, J=6 Hz), 4.05 (2H, m), 4.13 (1H, dd, J=7 and 3 Hz), 4.2 (1H, t, J=7 Hz), 4.4 (1H, t, J=3 Hz), 4.48 (1H, d, J=8 Hz), 4.6 (1H, t, J=3 Hz), 4.75 (1H, d, J=7 Hz), 4.82 (1H, d, J=3 Hz).

### EXAMPLE 3

### Preparation of the Carboxyl-Reduced and Persulfated Hyaluronic Acid Sodium Salt

(Polysaccharide of formula (I) with n=0 to 25 000, R═R1=SO₃Na) 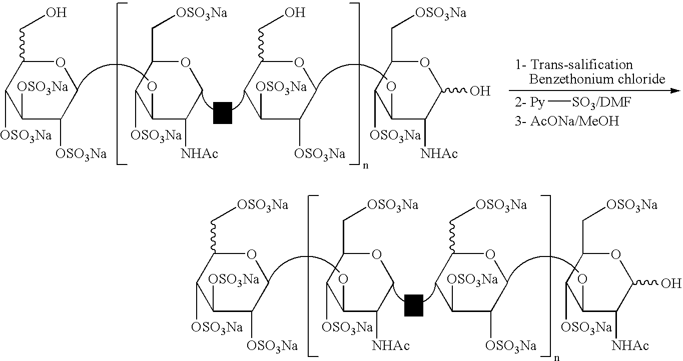
a) Preparation of the Persulfated, Carboxyl-Reduced Hyaluronic Acid Benzethonium Salt (Trans-Salification)

A solution of 0.507 g of benzethonium chloride in 10 ml of water is added to a solution of 0.218 g of carboxyl-reduced, persulfated hyaluronic acid sodium salt as described in example 2a or 2b, in 15 ml of water. The product is filtered, washed with water and dried. After drying for 48 h under reduced pressure (approximately 6 kPa) at a temperature in the region of 55° C., 0.453 g of a white solid is obtained. The yield obtained is 69%.

b) Preparation of the Carboxyl-Reduced, Persulfated Hyaluronic Acid Sodium Salt (Sulfatation)

0.45 g of persulfated, carboxyl-reduced hyaluronic acid benzethonium salt is dissolved in 35 ml of anhydrous dimethylformamide. A solution of 0.61 g of pyridine-sulfuric anhydride complex in 28 ml of anhydrous dimethylformamide is added at a temperature in the region of 20° C. and with stirring. After stirring for 3 hours at a temperature in the region of 20° C., a mixture of 32 ml of water and 190 ml of a 10% sodium acetate solution in methanol is added. The suspension is filtered. The cake is washed with 4 portions of 50 ml of methanol. The white solid obtained is dissolved in 10 ml of water and is then filtered through a 0.45 μm membrane. The filtrate is loaded into an MW 10 000 cellulose ester membrane and dialyzed, changing the water baths regularly for 24 hours. The content of the membranes is lyophilized. 0.197 g of a white lyophilized material is obtained. The yield obtained is quantitative.

Proton spectrum in D₂O, 400 MHz, T=303 K, δ in ppm: 2.1 (3H, s), between 3.8 and 4.0 (3H, m), 4.1 (1H, t, J=7 Hz), between 4.2 and 4.6 (5H, m), 4.6 (1H, d, J=6 Hz), between 4.68 and 4.8 (3H, m), 5.18 (1H, s).

Pharmacological Tests

Effect of the carboxyl-reduced derivatives on the aggrecanase in synovial fluids or on preparations of recombinant ADAMTS protein.

The analysis is carried out on 96-well plates. Before the analysis, serial dilutions of the assay compounds are prepared in an aqueous solution.

Digestion:

In each well, a fixed volume of synovial fluid or aggrecanase activity generating a known increase, between 1.0 and 1.4 absorbance units (405 nm), under the conditions of the analysis, is mixed with 3 μl of solution of compound from the respective dilution step. Dulbecco's modified Eagle's medium (DMEM) is added to each well to give a final volume of 300 μl. The plate is then incubated for 1 hour at 37° C. in a carbon dioxide atmosphere.

After the addition of 5 μl of a solution of 1 μg/∞l of recombinant substrate Agglmut in DMEM to each well (substrate as described by Bartnik E. et al., EP 785274, 1997), the reagent mixture is incubated for 4 hours at 37° C. under a carbon dioxide atmosphere.

Preparation of the Analytical Plate:

In a first step, the microplate is coated with 100 μl per well of a commercial goat anti-mouse IgG antibody for 1 hour at ambient temperature (5 μg/ml in a phosphate buffered saline solution, pH 7.4 [PBS buffer]). After washing with the PBS buffer containing 0.1% of Tween-20 (washing buffer), the wells are blocked by means of a step consisting of a one hour incubation with 100 μl per well of 5% bovine serum albumin in a PBS buffer containing 0.05% of Tween-20. Following another rinse with the washing buffer, each well is incubated with 100 μl of a solution, diluted to 1:1000, of BC-3 antibody in a PBS buffer containing 0.05% of Tween 20 and 0.5% of bovine serum albumin, at ambient temperature for one hour. This antibody recognizes the typical aggrecanase cleavage fragments (Hughes C. E. et al., Biochem. J. 305 (3), 799-804, 1995).

Assay Procedure:

After another rinse of the analytical plate with the washing buffer, the complete set of mixtures originating from the preceding digestion is transferred to this plate, well by well, and incubated at ambient temperature for one hour. The plate is again washed as above. Next, 100 μl of the second antibody (anti-human IgG antibody labeled with peroxidase, 1:1000 in PBS containing 0.5% of BSA and 0.05% of Tween-20) are added to each well, with subsequent incubation at ambient temperature again for one hour. After a final rinse with the washing buffer, the color development is initiated by adding 100 μl of ABTS substrate solution (2 mg/ml, 2,2′-azinobis(3-ethyl-benzothiazoline)sulfonic acid) in 40 mM of sodium citrate plus 60 mM of disodium hydrogen phosphate, adjusted to a pH of 4.4 by adding acetic acid; 0.25 μl of 35% hydrogen peroxide added per ml immediately before measurement). The measurement is carried out by means of the screen mode using detection at 405 nm relative to a reference filter (620 nm) with automatic readings at 5-second intervals. The development is stopped as soon as the maximum signal (405 nm) in the absorbance range of 1.0 to 1.4 is reached.

TABLE 1

| | |
|---|---|
| Concentration of | % conversion |

| glycosaminoglycan | Product | Product | product |
|---|---|---|---|
| (μg/ml) | example 2 | example 3 | example 1 |
| 10 | 17.4 | 21 | 100 |
| 1 | 16.9 | 27.4 | 100 |
| 0.1 | 17.0 | 28 | 100 |
| 0.01 | 47.6 | 54.3 | 100 |
| 0.001 | 88.9 | 84.9 | 100 |
| 0.0001 | 98 | 93 | 100 |
| IC₅₀ (μg/ml) | 0.0088 | 0.025 | no |
| for inhibition | | | inhibition |

In this test, the starting hyaluronic acid shows no aggrecanase-inhibiting activity.

Effect of the Persulfated, Carboxyl-Reduced Derivative (Example 2) on the Aggrecanase of Bovine Chondrocytes Cultured in Alginate Beads

In order to generate aggrecanase activity, the bovine chondrocytes cultured in gels of alginate matrix are stimulated with 10 ng/ml of IL-1α for 3 days in accordance with the method of C. E. Hughes et al., J. Biol. Chem. 273, 30576-30582, 1998.

In each well of a 96-well cell culture plate, 200 μl of chondrocyte supernatant containing aggrecanase activity are mixed with 100 μl of DMEM cell culture medium. At the concentrations to be tested, 5 μl of an aqueous solution of the product of example 2 is added in order to inhibit the aggrecanase activity, one hour before the addition of 5 μg of recombinant substrate Agglmut (E. Bartnik et al., EP 785274, 1997). The mixture is incubated at 37° C. for 17 hours and is then transferred into an ELISA plate in order to detect the neoepitopes generated by the aggrecanase activity, by binding antibody BC-3 as previously described (C. E. Hughes et al., Biochem. J. 305 (3), 799-804, 1995).

TABLE 2
(% inhibition of the conversion of the substrate by means
of the aggrecanase activity in the supernatant)

| Concentration of the | |
|---|---|
| product of example 2 | |
| (μg/ml) | % inhibition |
| 100 | 85 |
| 10 | 83.9 |
| 1 | 13.7 |
| 0.1 | 0 |
| 0.01 | 0 |

IC₅₀:0.0033 mg/ml

Inhibition of Factor Xa

For the calibration, a standard sample of low molecular weight heparin (Enoxaparin) was used as reference.

The serial dilutions of the assay compound are prepared in a 0.046 M Tris buffer, pH 8.4, containing 0.15 M NaCl, 0.007 M EDTA, 0.1% of Tween 80 and 0.12 IU/ml of human antithrombin III. The samples of 50 μl of the respective dilutions are incubated with 50 μl of bovine factor Xa (13.6 U/ml) at 37° C. for 80 seconds. Next, 50 μl of chromogenic substrate, 1.1 mM S-2765, are added. The absorbance at 405 nm is measured in a photometer. The activity of the deblocked factor Xa is indicated by the emission of p-nitroaniline from the substrate.

When compared to the low-molecular weight reference heparin compound (100 U/mg), the glycosaminoglycans present a factor Xa, with much lower inhibitory activity:
Product example 2: 0.485 U/mg
Product example 3: 1.75 U/mg
   Effect of the Carboxyl-Reduced Persulfate Derivative (Example 2) for Human Metalloproteases MMP-2 (Gelatinase-A) and MMP7

Commercial ELISA kits (such as those provided, for example, by the company Amersham Biosciences, kits RPN2617 and RPN2620) were used, respectively, to determine the inhibitory activity with respect to the MMP-2 and MMP-7 enzymes. The concentrations of the enzymes are, respectively, 800 ng/ml for MMP-2 and 300 ng/ml for MMP-7. The tests were carried out in accordance with the manufacturer's recommendations, by performing a series of dilutions of the compound to be studied in a PBS buffer, pH 7.5. The MMP-2 and MMP-7 enzymes were both inhibited in a concentration-dependent manner.

Derivative obtained according to example 2
IC_{50: 0.8} μg/ml (effect on MMP-2)
0.2 μg/ml (effect on MMP-7).
   Effects of the Carboxyl-Reduced Derivatives According to the Invention on Human Neutrophil Elastase

A commercially available enzyme (for example, human neutrophil elastase, sigma No. E8140) is reconstituted in aliquots of 0.1 mg per vial with 0.276 ml of 50 mM sodium acetate buffer, pH 5.5, containing 200 mM NaCl (stock solution of the enzyme); 11 μl of this stock solution are diluted with 1.1 ml of the HEPES buffer above (enzyme assay solution).

The substrate solution is prepared by dissolving 118 mg of methoxy-succinyl-L-Ala-L-Ala-L-Pro-L-Val-p-nitro-anilide in 1 ml of DMSO (stock solution, for example sigma No. M4765). In view of the application in the analysis, 9 μl of stock solution are then diluted in 1.19 ml of water.

The serial dilutions of the assay compounds are prepared in 100 mM of HEPES buffer containing 500 mM NaCl. The analysis is carried out on colorless 96-well polystyrene microplates (for example, Corning Costar, No. 3695).

10 μl of assay enzyme solution are mixed with 10 μl of the respective diluted compound solution and 10 μl of the substrate solution. After incubation for 15 min, the cleavage of the substrate is read as increase in absorbance at 405 nm.

The product of example 2 inhibits human neutrophil elastase with an IC₅₀ of 0.15 μg/ml.

The inhibition with the product of example 3 is significantly less, with an IC₅₀ of 7.0 μg/ml.

1. A carboxyl-reduced and chemoselectively O-sulfated compound of hyaluronic acid, as a single compound or as a mixture, or the salts thereof.

2. A carboxyl-reduced compound of hyaluronic acid as claimed in claim 1, of formula (I): 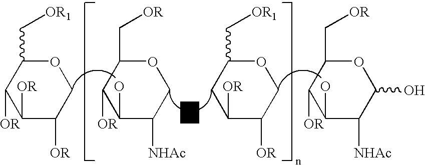
wherein R represents SO₃M, and R is H or SO₃M, n is an integer of between 0 and 25,000, M is an alkali metal, said compound isolated as a single compound or as a mixture, or the diastereoisomers thereof.

3. A carboxyl-reduced derivative of hyaluronic acid as claimed in claim 1, wherein M is chosen from sodium, calcium, magnesium and potassium.

4. A carboxyl-reduced derivative of hyaluronic acid as claimed in claim 3, wherein M is sodium.

5. A carboxyl-reduced derivative of hyaluronic acid as claimed in claim 4, wherein R and R₁ is SO₃Na.

6. A carboxyl-reduced derivative of hyaluronic acid as claimed in claim 4, wherein R is SO₃Na and R₁ is H.

7. A process for preparing the carboxyl-reduced derivatives of hyaluronic acid as claimed in claim 1 comprising the following steps:
trans-salifying of hyaluronic acid with a quaternary ammonium salt,
sulfating in organic medium of the trans-salified hyaluronic acid,
salifying the persulfated hyaluronic acid
reducing the carboxyl group of the persulfated hyaluronic acid
optionally a) by reducing a carbodiimide adduct of the carboxyl group in the presence of a reducing agent,
or b) by esterifying, the persulfated compound being, where appropriate, trans-salified beforehand with a quaternary ammonium salt,
reducing the esterified persulfated compound with a reducing agent; and
where appropriate, trans-salifying the persulfated, carboxyl-reduced derivative with a quaternary ammonium salt, and then resulfating followed by salifying.

8. The process as claimed in claim 7, wherein the sulfating is carried out in organic medium by means of a complex of sulfuric anhydride with an organic base chosen from pyridine and trimethylamine.

9. The process as claimed in claim 7, wherein the hyaluronic acid quarternary salt to be sulfated is hyaluronic benzethonium salt, sulfating said salt in the presence of about 10 to about 30 equivalents of pyridine-sulfuric anhydride complex per hydroxyl function to be sulfated, and at a temperature in the range of between about 10° C. and about 70° C.

10. The process as claimed in claim 9 wherein the temperature is in the range of between about 50° C. and about 70° C.

11. The process as claimed in claim 7, further comprising reducing the carboxyl in the presence of 1-(3-dimethyl-aminopropyl)-3-ethylcarbodiimide hydrochloride with an alkali metal borohydride.

12. The process as claimed in claim 11, wherein the amount of 1-(3-dimethylaminopropyl)-3-ethylcarbodiimide hydrochloride used is within the range of from about 5 equivalents to about 20 equivalents per carboxyl and at a pH of between 4 and 5.

13. The process as claimed in claim 11 wherein the alkali metal borohydride is sodium borohydride.

14. The process as claimed in claim 12, wherein 7 to 13 equivalents of 1-(3-dimethylaminopropyl)-3-ethylcarbodiimide hydrochloride are used, and at a pH of between about 4.3 and about 4.9.

15. The process as claimed in claim 11, wherein the reduction of the activated adduct of the hyaluronic acid derivative is carried out with about 10 to about 300 equivalents of alkali metal borohydride at a temperature range of between about 10° C. and about 70 C.

16. The process as claimed in claim 15, wherein the reduction of the adduct is carried out with about 140 to about 250 equivalents of alkali metal borohydride at a temperature range of between about 20° C. and about 50° C.

17. The process as claimed in claim 7, wherein the ester to be reduced is a methyl ester and said reducing agent is an alkali metal borohydride.

18. The process as claimed in claim 17, further comprising preparing said methyl ester from benzethonium salt of hyaluronic acid, in a solution in dichloromethane with an amount of about 2 to about 20 equivalents of methyl iodide per equivalent of benzethonium salt of hyaluronic acid.

19. The process as claimed in claim 18, wherein the amount of methyl iodide is in the range of about 4 to about 10 equivalents.

20. The process as claimed in claim 17, wherein the reduction of the ester of the hyaluronic acid compound is carried out with about 10 to about 300 equivalents of alkali metal borohydride at a temperature in the range of between about 10° C. and about 50° C.

21. The process as claimed in claim 20, wherein the reduction of the ester is carried out in the presence of about 140 to about 250 equivalents of alkali metal borohydride at a temperature of about 20° C.

22. The process as claimed in claim 21 wherein the alkali metal borohydride is sodium borohydride.

23. The process as claimed in claim 7, for obtaining the derivatives of formula (I) as defined in claim 5, wherein the sulfation of the benzethonium salt of the persulfated, carboxyl-reduced derivative as defined in claim 6 is carried out in organic medium by means of a complex of sulfuric anhydride with an organic base chosen from pyridine and trimethylamine, at about 20° C.

24. The process as claimed in claim 7, further comprising the following steps:
trans-salifying of hyaluronic acid with benzethonium chloride,
sulfating of a quaternary ammonium salt of hyaluronic acid in organic medium by means of a complex of sulfuric anhydride with pyridine or trimethylamine, followed by salifying with sodium acetate,
optionally a) reducing the carboxyl group in the presence of 1-(3-dimethylaminopropyl)-3-ethylcarbodiimide hydrochloride with sodium borohydride,
or b) esterifying by reacting methyl iodide with the hyaluronic acid compound trans-salified beforehand with benzethonium chloride,
reducing the corresponding methyl ester with sodium borohydride; and
where appropriate, trans-salifying the sulfated, carboxyl-reduced derivative with benzethonium chloride, and then resulfating with the sulfuric anhydride-organic base complex, followed by salifying with sodium acetate.

25. A process for obtaining the isolated carboxyl-reduced and chemoselectively O-sulfated compounds of hyaluronic acid as claimed in claim 1, from the mixture of carboxyl-reduced compounds of hyaluronic acid, further comprising fractionating said mixture by column chromatography on columns filled with polyacrylamide agarose gel or polyacrylamide gel, said mixture being eluted with a sodium hydrogen carbonate solution, in a concentration of about 0.1 to about 1 mol/liter.

26. A pharmaceutical composition comprising a compound of claim 1 and one or more pharmaceutically acceptable excipients.

27. A method of treating a disease in a patient characterized by an increased activity in at least one of the matrix metalloproteinases selected from the group consisting of neutrophil elastase, matrilysin (MMP-7), aggrecanase hADAMTS1 and gelatinase A (MMP-2), comprising administering to said patient a therapeutically effective amount of a compound as claimed in claim 1.

28. The method of claim 27 wherein the disease is selected form the group consisting of joint degeneration, spondylosis, chondrolysis associated with joint trauma or prolonged immobilization of the joint, connective tissue disorders, wound healing conditions, periodontal disorders, chronic disorders of the locomotor system, arthropathies, myalgias and bone metabolism disorders.
